(19) United States
(12) Patent Application Publication
(10) Pub. No.: US 2013/0190506 A1
(43) Pub. Date: Jul. 25, 2013

(54) PROCESS FOR OLMESARTAN MEDOXOMIL

(71) Applicants: Bandi Parthasaradhi Reddy, Andhra Pradesh (IN); Kura Rathnakar Reddy, Andhra Pradesh (IN); Dasari Muralidhara Reddy, Andhra Pradesh (IN); Rapolu Raji Reddy, Andhra Pradesh (IN); Matta Ramakrishna Reddy, Andhra Pradesh (IN); Bandi Vamsi Krishna, Andhra Pradesh (IN)

(72) Inventors: Bandi Parthasaradhi Reddy, Andhra Pradesh (IN); Kura Rathnakar Reddy, Andhra Pradesh (IN); Dasari Muralidhara Reddy, Andhra Pradesh (IN); Rapolu Raji Reddy, Andhra Pradesh (IN); Matta Ramakrishna Reddy, Andhra Pradesh (IN); Bandi Vamsi Krishna, Andhra Pradesh (IN)

(73) Assignee: HETERO RESEARCH FOUNDATION, Andhra Pradesh (IN)

(21) Appl. No.: 13/805,904

(22) PCT Filed: Jun. 28, 2010
(86) PCT No.: PCT/IN2010/000441

§ 371 Date: Apr. 8, 2013

(51) Int. Cl. C07D 405/14

(52) U.S. Cl. CPC C07D 405/14
USPC 548/253

(57) ABSTRACT

The present invention provides a process for the preparation of substantially pure trityl olmesartan medoxomil. The present invention also provides a process for purification of trityl olmesartan medoxomil. The present invention further provides a process for purification of olmesartan medoxomil.

### FIELD OF THE INVENTION

The present invention provides a process for the preparation of substantially pure trityl olmesartan medoxomil. The present invention also provides a process for purification of trityl olmesartan medoxomil. The present invention further provides a process for purification of olmesartan medoxomil.

### BACKGROUND OF THE INVENTION

Olmesartan medoxomil is chemically, 4-(1-hydroxy-1-methylethyl)-2-propyl-1-[[2′-(1H-tetrazol-5-yl) [1,1′-biphenyl]-4-yl]methyl]-1H-imidazole-5-carboxylic acid (5-methyl-2-oxo-1,3-dioxol-4-yl)methyl ester. Olmesartan medoxomil is represented by the following structure.

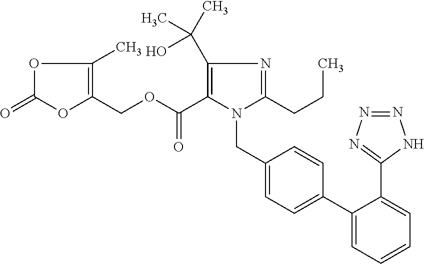 [SMILES: CCCc1nc(C(C)(C)O)c(C(=O)OCc2oc(=O)oc2C)n1Cc1ccc(-c2ccccc2-c2nnn[nH]2)cc1]

Olmesartan medoxomil is prodrug that is hydrolyzed during absorption, and it is a selective AT₁ subtype angiotensin II receptor antagonist. Olmesartan medoxomil is disclosed by U.S. Pat. No. 5,616,599. It is marketed as Benicar® in film-coated tablets of 5 mg, 20 mg, and 40 mg for treatment of hypertension in a human.

EP patent application no. 1916246 disclosed a process for the preparation of olmesartan medoxomil which comprises reacting trityl olmesartan medoxomil with aqueous acetic acid to give olmesartan medoxomil, which is further, crystallized using isopropyl alcohol followed by purification from methyl ethyl ketone to give substantially pure olmesartan medoxomil.

PCT publication no. WO 2006/073518 described a process for the preparation of dissolving trityl olmesartan medoxomil in a mixture of an organic solvent such as acetonitrile, isopropyl alcohol or t-butanol and water to form solution has a pH of at least 2.5 and heating the solution to obtain olmesartan medoxomil.

PCT publication no. WO 2009/019303 disclosed a process for the purification of olmesartan medoxomil which comprises:
a) providing protected olmesartan medoxomil and forming the solution containing the protected olmesartan medoxomil and hydrobromic acid in a mixture of water miscible organic solvent such as acetone or tetrahydrofuran and water, thereby removing the protecting group; or
b) forming a solution containing olmesartan medoxomil and hydrobromic acid; and
c) forming olmesartan medoxomil hydrobromide salt in solid form and isolating the olmesartan medoxomil hydrobromide salt; and optionally
d) converting the olmesartan medoxomil hydrobromide salt to olmesartan medoxomil.

U.S. patent application no. 2007/0105923 disclosed a process for the purifying olmesartan medoxomil which comprises dissolving olmesartan medoxomil in a solvent system comprising acetone and at least one solvent selected from ethyl acetate, isopropyl alcohol and mixtures thereof to obtain a solution; and recovering substantially pure olmesartan medoxomil.

U.S. patent application no. 2006/0074117 disclosed a process for purifying olmesartan medoxomil which comprises providing a solution of olmesartan medoxomil in acetone and adding water to the solution; and recovering purified olmesartan medoxomil.

The potential impurities of the trityl olmesartan medoxomil are ethyl-4-(1-hydroxy-1-ethylmethyl)-2-propyl-1-[[2′-(N-triphenylmethyl-1H-tetrazole-5-yl)[1,1′-biphenyl]-4-yl]methyl-1H-imidazole-5-carboxylate, 4-(1-hydroxy-1-methylethyl)-2-propyl-1-[[2′-(N-triphenylmethyl-1H-tetrazol-5-yl) [1,1′-biphenyl]-4-yl]methyl]-1H-imidazole-5-carboxylicacid (5-bromo-2-oxo-1,3-dioxal-4-yl)methylester and 4-(1-methoxy-1-methylethyl)-2-propyl-1-[[2′-(N-triphenylmethyl-1H-tetrazol-5-yl)[1,1′-biphenyl]-4-yl]methyl]-1H-imidazole-5-carboxylicacid (5-methyl-2-oxo-1,3-dioxal-4-yl)methylester.

The chemical formula of ethyl-4-(1-hydroxy-1-ethylmethyl)-2-propyl-1-[[2′-(N-triphenylmethyl-1H-tetrazole-5-yl)[1,1′-biphenyl]-4-yl]methyl-1H-imidazole-5-carboxylate (trityl olmesartan ethyl ester impurity) may be represented as:

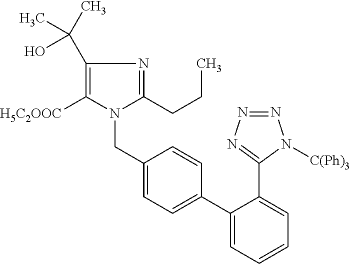

The chemical formula of 4-(1-hydroxy-1-methylethyl)-2-propyl-1-[[2′-(N-triphenylmethyl-1H-tetrazol-5-yl)[1,1′-biphenyl]-4-yl]methyl]-1H-imidazole-5-carboxylicacid (5-bromo-2-oxo-1,3-dioxal-4-yl)methylester (bromo trityl olmesartan medoxomil impurity) may be represented as:

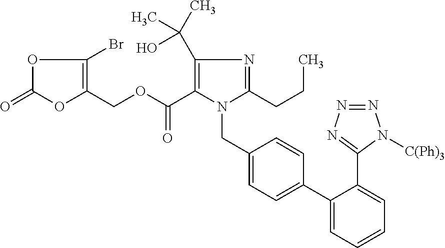

The chemical formula of 4-(1-methoxy-1-methylethyl)-2-propyl-1-[[2′-(N-triphenylmethyl-1H-tetrazol-5-yl) [1,1′-biphenyl]-4-yl]methyl]-1H-imidazole-5-carboxylicacid (5-methyl-2-oxo-1,3-dioxal-4-yl)methylester (methyl trityl olmesartan medoxomil impurity) may be represented as:

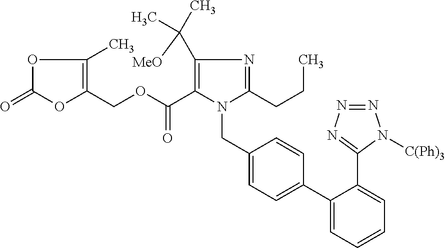

We have found a simple and effective process for the reduction of ethyl-4-(1-hydroxy-1-ethylmethyl)-2-propyl-1-[[2′-(N-triphenylmethyl-1H-tetrazole-5-yl)[1,1′-biphenyl]-4-yl]methyl-1H-imidazole-5-carboxylate, 4-(1-hydroxy-1-methylethyl)-2-propyl-1-[[2′-(N-triphenylmethyl-1H-tetrazol-5-yl)[1,1′-biphenyl]-4-yl]methyl]-1H-imidazole-5-carboxylicacid (5-bromo-2-oxo-1,3-dioxal-4-yl)methylester and 4-(1-methoxy-1-methylethyl)-2-propyl-1-[[2′-(N-triphenylmethyl-1H-tetrazol-5-yl)[1,1′-biphenyl]-4-yl]methyl]-1H-imidazole-5-carboxylicacid (5-methyl-2-oxo-1,3-dioxal-4-yl)methylester impurities in the trityl olmesartan medoxomil.

The potential impurities of the olmesartan medoxomil are 4-(1-hydroxy-1-methylethyl)-2-propyl-1-[[2′-(1H-tetrazole-5-yl)[1,1′-biphenyl]-4-yl]methyl]-1H-imidazole-5-carboxylic acid, 4-(1-hydroxy-1-methylethyl)-2-propyl-1-[[2-′(N-triphenylmethyl-1H-tetrazol-5-yl)[1,1′-biphenyl]-4-yl]methyl]-1H-imidazole-5-carboxylicacid (5-methyl-2-oxo-1,3-dioxal-4-yl)methylester and 4-(1-methoxy-1-methylethyl)-2-propyl-1-[[2′-(1H-tetrazole-5-yl)[1,1′-biphenyl]-4-yl]methyl]-1H-imidazole-5-carboxylic acid (5-methyl-2-oxo-1,3-dioxal-4-yl)methylester.

The chemical formula of 4-(1-hydroxy-1-methylethyl)-2-propyl-1-[[2′-(1H-tetrazole-5-yl)[1,1′-biphenyl]-4-yl]methyl]-1H-imidazole-5-carboxylic acid (olmesartan acid impurity) may be represented as:

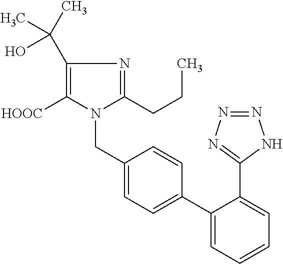 [SMILES: CCCc1nc(C(C)(C)O)c(C(=O)O)n1Cc1ccc(-c2ccccc2-c2nnn[nH]2)cc1]

The chemical formula of 4-(1-hydroxy-1-methylethyl)-2-propyl-1-[[2-′(N-triphenylmethyl-1H-tetrazol-5-yl)[1,1′-biphenyl]-4-yl]methyl]-1H-imidazole-5-carboxylicacid (5-methyl-2-oxo-1,3-dioxal-4-yl)methylester (trityl olmesartan medoxomil impurity) may be represented as:

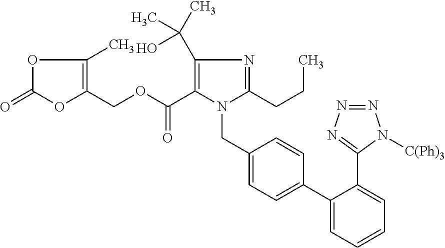

The chemical formula of 4-(1-methoxy-1-methylethyl)-2-propyl-1-[[2′-(1H-tetrazole-5-yl)[1,1′-biphenyl]-4-yl]methyl]-1H-imidazole-5-carboxylic acid (5-methyl-2-oxo-1,3-dioxal-4-yl)methylester (methyl trityl olmesartan medoxomil impurity) may be represented as:

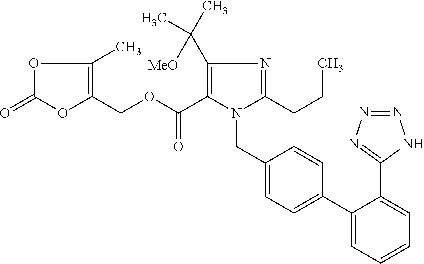 [SMILES: CCCc1nc(C(C)(C)OC)c(C(=O)OCc2oc(=O)oc2C)n1Cc1ccc(-c2ccccc2-c2nnn[nH]2)cc1]

We have also found a simple and effective process for the reduction of 4-(1-hydroxy-1-methylethyl)-2-propyl-1-[[2′-(1H-tetrazole-5-yl)[1,1′-biphenyl]-4-yl]methyl]-1H-imidazole-5-carboxylic acid, 4-(1-hydroxy-1-methylethyl)-2-propyl-1-[[2-′(N-triphenylmethyl-1H-tetrazol-5-yl)[1,1′-biphenyl]-4-yl]methyl]-1H-imidazole-5-carboxylicacid (5-methyl-2-oxo-1,3-dioxal-4-yl)methylester and 4-(1-methoxy-1-methylethyl)-2-propyl-1-[[2′-(1H-tetrazole-5-yl)[1,1′-biphenyl]-4-yl]methyl]-1H-imidazole-5-carboxylic acid (5-methyl-2-oxo-1,3-dioxal-4-yl)methylester impurities in the olmesartan medoxomil.

Thus, one object of the present invention is to provide a process for the preparation of substantially pure trityl olmesartan medoxomil.

Another object of the present invention is to provide a process for the purification of trityl olmesartan medoxomil.

Yet another object of the present invention is to provide a process for the purification of olmesartan medoxomil.

### DETAILED DESCRIPTION OF THE INVENTION

According to one aspect of the present invention, there is provided a process for the preparation of substantially pure 4-(1-hydroxy-1-methylethyl)-2-propyl-1-[[2′-(N-triphenylmethyl-1H-tetrazole-5-yl)[1,1′-biphenyl]-4-yl]methyl]-1H-imidazole-5-carboxylic acid (5-methyl-2-oxo-1,3-dioxal-4-yl)methyl ester (trityl olmesartan medoxomil), which comprises adding a solution of 4-(1-hydroxy-1-methylethyl)-2-propyl-1-[[2′-(N-triphenylmethyl-1H-tetrazole-5-yl)[1,1′-biphenyl]-4-yl]methyl]-1H-imidazole-5-carboxylic acid (trityl olmesartan acid) in a solvent selected from ketonic solvent, dimethylformamide and dimethyl acetate to a solution of (4-bromoomethyl)-5-methyl-2-oxo-1,3-dioxane (medoxomil) in a solvent selected from ketonic solvent, dimethylformamide and dimethyl acetate to obtain substantially pure trityl olmesartan medoxomil.

The term “substantially pure trityl olmesartan medoxomil” refers to olmesartan medoxomil having the purity greater than about 85% by weight, preferably greater than about 90% by weight, more preferably greater than about 95% by weight.

Preferably the solvent used in the process is ketonic solvent selected from acetone, methyl ethyl ketone, methyl isobutyl ketone and diethyl ketone, and more preferable ketonic solvent is acetone.

The reaction is preferably carried out at about below 65° C. and more preferably at about 35 to 65° C., still more preferably at about 45 to 55° C.

Substantially pure trityl olmesartan medoxomil obtained as per this process containing low level of impurities particularly, ethyl-4-(1-hydroxy-1-ethylmethyl)-2-propyl-1-[[2′-(N-triphenylmethyl-1H-tetrazole-5-yl)[1,1′-biphenyl]-4-yl]methyl-1H-imidazole-5-carboxylate, 4-(1-hydroxy-1-methylethyl)-2-propyl-1-[[2′-(N-triphenylmethyl-1H-tetrazol-5-yl)[1,1′-biphenyl]-4-yl]methyl]-1H-imidazole-5-carboxylicacid (5-bromo-2-oxo-1,3-dioxal-4-yl)methylester and 4-(1-methoxy-1-methylethyl)-2-propyl-1-[[2′-(N-triphenylmethyl-1H-tetrazol-5-yl) [1,1′-biphenyl]-4-yl]methyl]-1H-imidazole-5-carboxylicacid (5-methyl-2-oxo-1,3-dioxal-4-yl)methylester.

According to another aspect of the present invention, there is provided a process for the purification of trityl olmesartan medoxomil, which comprises:
a. stirring trityl olmesartan medoxomil with a solvent system comprising a solvent selected from ether solvent, ester solvent and mixture thereof; and water; and
b. isolating highly pure trityl olmesartan medoxomil.

The term “highly pure trityl olmesartan medoxomil” refers to olmesartan medoxomil having the purity greater than about 98% by weight, preferably greater than about 99% by weight, more preferably greater than about 99.5% by weight.

The ether solvent used in step (a) may preferably be selected from methyl tert-butyl ether, tetrahydrofuran, 1,4-dioxane and diethyl ether, and more preferable ether solvent is methyl tert-butyl ether.

The ester solvent used in step (a) may preferably be selected from ethyl acetate, methyl acetate, isopropyl acetate, tert-butyl methyl acetate and ethyl formate, and more preferable ester solvent is ethyl acetate.

The step (a) is preferably carried out at elevated temperature. The term “elevated temperature” refers to temperature at above 25° C. More preferably the step (a) is carried out at about 40 to 90° C. and still more preferably at about 45 to 80° C.

Preferably water to the solvent is between 4:1 and 1:1, and more preferably between 3:1 and 1.5:1.

Isolation of highly pure trityl olmesartan medoxomil may preferably be carried out by methods known such as filtration or centrifugation.

The purification process yields trityl olmesartan medoxomil with reduced levels of impurities, specifically, ethyl-4-(1-hydroxy-1-ethylmethyl)-2-propyl-1-[[2′-(N-triphenylmethyl-1H-tetrazole-5-yl) [1,1′-biphenyl]-4-yl]methyl-1H-imidazole-5-carboxylate, 4-(1-hydroxy-1-methylethyl)-2-propyl-1-[[2′-(N-triphenylmethyl-1H-tetrazol-5-yl)[1,1′-biphenyl]-4-yl]methyl]-1H-imidazole-5-carboxylicacid (5-bromo-2-oxo-1,3-dioxal-4-yl)methylester and 4-(1-methoxy-1-methylethyl)-2-propyl-1-[[2′-(N-triphenylmethyl-1H-tetrazol-5-yl) [1,1′-biphenyl]-4-yl]methyl]-1H-imidazole-5-carboxylicacid (5-methyl-2-oxo-1,3-dioxal-4-yl)methylester.

According to another aspect of the present invention, there is provided a process for the purification of olmesartan medoxomil, which comprises:
a. stirring a suspension or a solution of olmesartan medoxomil with methyl ethyl ketone; and
b. isolating highly pure olmesartan medoxomil.

The term “highly pure olmesartan medoxomil” refers to olmesartan medoxomil having the purity greater than about 98% by weight, preferably greater than about 99% by weight, more preferably greater than about 99.5% by weight.

The step (a) of the suspension or solution is preferably carried out at elevated temperature. The term “elevated temperature” refers to temperature at above 25° C. and more preferably carried out at about 30° C. to reflux temperature of methyl ethyl ketone.

Isolation of highly pure olmesartan medoxomil may preferably be carried out by methods known such as filtration or centrifugation.

The purification process yields olmesartan medoxomil with reduced levels of impurities, specifically, 4-(1-hydroxy-1-methylethyl)-2-propyl-1-[[2′-(1H-tetrazole-5-yl)[1,1′-biphenyl]-4-yl]methyl]-1H-imidazole-5-carboxylic acid, 4-(1-hydroxy-1-methylethyl)-2-propyl-1-[[2-′(N-triphenylmethyl-1H-tetrazol-5-yl) [1,1′-biphenyl]-4-yl]methyl]-1H-imidazole-5-carboxylicacid (5-methyl-2-oxo-1,3-dioxal-4-yl)methylester and 4-(1-methoxy-1-methylethyl)-2-propyl-1-[[2′-(1H-tetrazole-5-yl) [1,1′-biphenyl]-4-yl]methyl]-1H-imidazole-5-carboxylic acid (5-methyl-2-oxo-1,3-dioxal-4-yl)methylester.

The purity of trityl olmesartan medoxomil and olmesartan medoxomil was measured by High performance liquid chromatography (HPLC).

The invention will now be further described by the following examples, which are illustrative rather than limiting.

### EXAMPLES

### Example 1

### Preparation of Trityl Olmesartan Medoxomil

Ethyl-4-(1-hydroxy-1-methylethyl)-2-propyl-imidazole-5-carboxylate (100 gm) was dissolved in acetone (2500 ml) and then added potassium carbonate (100 gm), 5-[4′-(bromomethyl)[1,1′-biphenyl]-2-yl]-2-(triphenylmethyl)-1H-tetrazole (250 gm) and tert-butyl ammonium bromide (15 gm) under stirring at room temperature. The temperature of the reaction mass was raised to 50 to 55° C. and maintained for 15 hours at 50 to 55° C. The reaction mass was cooled to 45° C. and passed over celite bed. The collected filtrate was cooled to 0 to 5° C. and then added a solution of potassium carbonate (36 gm) in water (36 ml) for 1 hour. The temperature of the reaction mass was raised to room temperature and maintained for 16 hours at room temperature. The acetone was distilled off completely under vacuum at below 40° C. to obtain residue. To the residue was added sodium chloride solution (10%, 900 ml) and then added ethyl acetate (1500 ml). The layers were separated and the aqueous layer was extracted. Combined the both organic layers and dried over sodium sulfate. The solvent was distilled off completely to obtain a residual mass. A mixture of acetone (1200 ml), potassium carbonate (100 gm), (4-bromoethyl)-5-methyl-oxo-1,3-dioxane (105 gm) and potassium iodide (17 gm) were added under stirring at room temperature and then the contents were heated to 50 to 55° C. The solution was added to the above residual mass for 1 hour 30 minutes and maintained for 1 hour 30 minutes at 50 to 55° C. The reaction mass was cooled to 45° C. and filtered. The solvent was distilled off completely to obtain residue. Toluene (1500 ml) was added to the residue and the layers were separated. The toluene layer was dried over sodium sulfate and distilled off the layer under vacuum up to obtain clear residual mass. To the residual mass was added methanol (1500 ml) and stirred for 30 minutes at room temperature. The reaction mass was cooled to 10 to 15° C. and maintained for 1 hour 30 minutes. The separated solid was filtered and dried at 40 to 45° C. for 7 hours to obtain 270 gm of trityl olmesartan medoxomil.

Trityl olmesartan medoxomil: 98.5%;
Trityl olmesartan ethyl ester impurity: 0.35%;
Bromo trityl olmesartan medoxomil impurity: 0.35%;
Methyl trityl olmesartan medoxomil impurity: 0.34%.

### Example 2

### Purification of Trityl Olmesartan Medoxomil

Trityl olmesartan medoxomil (260 gm; HPLC Purity: 98.5%) as obtained in example 1 was added to ethyl acetate (1300 ml) and water (2600 ml) at room temperature. The temperature of the reaction mass was raised to 70 to 75° C. and stirred for 20 minutes at 70 to 75° C. The reaction mass was cooled to room temperature and stirred for 30 minutes at room temperature. The reaction mass was further cooled to 0 to 5° C. and stirred for 1 hour at 0 to 5° C. The solid obtained was collected by filtration and dried at 40 to 45° C. for 7 hours to obtain 250 gm of highly pure trityl olmesartan medoxomil.

Trityl olmesartan medoxomil: 99.65%;
Trityl olmesartan ethyl ester impurity: 0.1%;
Bromo trityl olmesartan medoxomil impurity: 0.12%;
Methyl trityl olmesartan medoxomil impurity: 0.1%.

### Example 3

Purification of Trityl Olmesartan Medoxomil

Trityl olmesartan medoxomil (100 gm; HPLC Purity: 98.5%) was added to ethyl acetate (600 ml) and water (1200 ml) at room temperature. The temperature of the reaction mass was raised to 70 to 75° C. and stirred for 20 minutes at 70 to 75° C. The reaction mass was cooled to room temperature and stirred for 30 minutes at room temperature. The reaction mass was further cooled to 0 to 5° C. and stirred for 1 hour at 0 to 5° C. The solid obtained was collected by filtration and dried at 40 to 45° C. for 7 hours to obtain 90 gm of highly pure trityl olmesartan medoxomil.

Trityl olmesartan medoxomil: 99.5%;
Trityl olmesartan ethyl ester impurity: 0.12%;
Bromo trityl olmesartan medoxomil impurity: 0.15%;
Methyl trityl olmesartan medoxomil impurity: 0.1%.

### Example 4

### Purification of Trityl Olmesartan Medoxomil

Trityl olmesartan medoxomil (50 gm; HPLC Purity: 98.5%) was added to ethyl acetate (250 ml) and water (520 ml) at room temperature. The temperature of the reaction mass was raised to 70 to 75° C. and stirred for 20 minutes at 70 to 75° C. The reaction mass was cooled to room temperature and stirred for 30 minutes at room temperature. The reaction mass was further cooled to 0 to 5° C. and stirred for 1 hour at 0 to 5° C. The solid obtained was collected by filtration and dried at 40 to 45° C. for 7 hours to obtain 46 gm of highly pure trityl olmesartan medoxomil.

Trityl olmesartan medoxomil: 99.55%;
Trityl olmesartan ethyl ester impurity: 0.1%;
Bromo trityl olmesartan medoxomil impurity: 0.15%;
Methyl trityl olmesartan medoxomil impurity: 0.12%.

### Example 5

### Purification of Trityl Olmesartan Medoxomil

Trityl olmesartan medoxomil (270 gm; HPLC Purity: 98.5%) was added to methyl tert-butyl ether (1350 ml) at room temperature. To the reaction mass was added water (2700 ml) under stirring and temperature of the reaction mass was raised to 50 to 55° C. The reaction mass was stirred for 45 minutes at 50 to 55° C. and the mass was cooled to room temperature. The reaction mass was further cooled to 10 to 15° C. and stirred for 1 hour at 10 to 15° C., filtered. The solid obtained was dried at 40 to 45° C. for 6 hours to obtain 260 gm of highly pure trityl olmesartan medoxomil.

Trityl olmesartan medoxomil: 99.7%;
Trityl olmesartan ethyl ester impurity: 0.08%;
Bromo trityl olmesartan medoxomil impurity: 0.1%;
Methyl trityl olmesartan medoxomil impurity: 0.08%.

### Example 6

### Purification of Trityl Olmesartan Medoxomil

Trityl olmesartan medoxomil (100 gm; HPLC Purity: 98.5%) was added to methyl tert-butyl ether (700 ml) at room temperature. Water (1400 ml) was added to the reaction mass under stirring and temperature of the reaction mass was raised to 50 to 55° C. The reaction mass was stirred for 45 minutes at 50 to 55° C. and the mass was cooled to room temperature. The reaction mass was further cooled to 10 to 15° C. and stirred for 1 hour at 10 to 15° C., filtered. The solid obtained was dried at 40 to 45° C. for 6 hours to obtain 92 gm of highly pure trityl olmesartan medoxomil.

Trityl olmesartan medoxomil: 99.6%;
Trityl olmesartan ethyl ester impurity: 0.1%;
Bromo trityl olmesartan medoxomil impurity: 0.11%;
Methyl trityl olmesartan medoxomil impurity: 0.12.

### Example 7

### Preparation of Olmesartan Medoxomil

Trityl olmesartan medoxomil (260 gm) as obtained in example 1 was dissolved in toluene (2600 ml) and then added concentrated hydrochloric acid (156 ml) for 1 hour minutes at room temperature. The reaction mass was maintained for 1 hour 30 minutes at room temperature and then added water (1000 ml). The reaction mass was stirred for 45 minutes at room temperature and the layers were separated. To the aqueous layer was added ethyl acetate (5000 ml) at room temperature. The reaction mass was cooled to 15 to 20° C. and pH of the reaction mass was adjusted to 4.5 to 5.5 with sodium carbonate (20%, 560 ml). The reaction mass was stirred for 20 minutes at 20° C. and the layers were separated. The organic layer was dried over sodium sulfate and ethyl acetate was distilled off completely under vacuum at below 45° C. to obtain a residual mass. To the residual mass was added ethyl acetate (400 ml) at 40° C. and then heated to 75 to 80° C. The contents were maintained for 30 minutes at 75 to 80° C. The reaction mass was cooled to room temperature and stirred for 1 hour. The reaction mass was further cooled to 10 to 15° C. and stirred for 1 hour 30 minutes, filtered. The solid obtained was dried at 40 to 45° C. for 4 hours to obtain 150 gm of olmesartan medoxomil.

Olmesartan medoxomil: 98.6%;
Olmesartan acid impurity: 0.32%;
Trityl olmesartan medoxomil impurity: 0.35%
Methyl olmesartan medoxomil impurity: 0.35%.

### Example 8

### Purification of Olmesartan Medoxomil

Olmesartan medoxomil (50 gm; HPLC Purity: 98.6%) as obtained in example 7 was added to methyl ethyl ketone (1100 ml) at room temperature and then the contents were heated to reflux. The reaction mass was treated with activated carbon to obtain solution and passed over hi-flo bed, and collected the filtrate. 50 percent of the methyl ethyl ketone volume was distilled off under vacuum at 45° C. The contents were heated to reflux and maintained for 30 minutes at reflux. The reaction mass was cooled to room temperature and stirred for 1 hours at room temperature. The reaction mass was further cooled to 10 to 15° C. and stirred for 1 hour 30 minutes at 0 to 5° C. The solid obtained was collected by filtration and dried at 50 to 55° C. for 7 hours to obtain 143 gm of highly pure olmesartan medoxomil.

Olmesartan medoxomil: 99.6%;
Olmesartan acid impurity: 0.07%;
Trityl olmesartan medoxomil impurity: 0.08%
Methyl olmesartan medoxomil impurity: 0.1%.

### Example 9

### Purification of Olmesartan Medoxomil

Olmesartan medoxomil (150 gm; HPLC Purity: 98.6%) was added to methyl ethyl ketone (750 ml) at room temperature. The contents were heated to reflux and then the reaction mass was treated with activated carbon to obtain solution. The solution passed over hi-flo bed and collected the filtrate. 50 percent of the methyl ethyl ketone volume was distilled off under vacuum at 45° C. The contents were heated to reflux and maintained for 30 minutes at reflux. The reaction mass was cooled to room temperature and stirred for 1 hours at room temperature. The reaction mass was further cooled to 10 to 15° C. and stirred for 1 hour 30 minutes at 0 to 5° C. The solid obtained was collected by filtration and dried at 50 to 55° C. for 7 hours to obtain 140 gm of highly pure olmesartan medoxomil.

Olmesartan medoxomil: 99.5%;
Olmesartan acid impurity: 0.09%;
Trityl olmesartan medoxomil impurity: 0.1%
Methyl olmesartan medoxomil impurity: 0.1%.

### Example 10

### Purification of Olmesartan Medoxomil

Olmesartan medoxomil (150 gm; HPLC Purity: 98.6%) was added to methyl ethyl ketone (1500 ml) at room temperature. The contents were heated to reflux and then the reaction mass was treated with activated carbon to obtain solution. The solution passed over hi-flo bed and collected the filtrate. 50 percent of the methyl ethyl ketone volume was distilled off under vacuum at 45° C. The contents were heated to reflux and maintained for 30 minutes at reflux. The reaction mass was cooled to room temperature and stirred for 1 hours at room temperature. The reaction mass was further cooled to 10 to 15° C. and stirred for 1 hour 30 minutes at 0 to 5° C. The solid obtained was collected by filtration and dried at 50 to 55° C. for 7 hours to obtain 142 gm of highly pure olmesartan medoxomil.

Olmesartan medoxomil: 99.55%;
Olmesartan acid impurity: 0.08%;
Trityl olmesartan medoxomil impurity: 0.1%
Methyl olmesartan medoxomil impurity: 0.09%.

### Example 11

### Purification of Olmesartan Medoxomil

Olmesartan medoxomil (150 gm; HPLC Purity: 98.6%) was added to methyl ethyl ketone (3400 ml) at room temperature. The contents were heated to reflux and then the reaction mass was treated with activated carbon to obtain solution. The solution passed over hi-flo bed and collected the filtrate. 50 percent of the methyl ethyl ketone volume was distilled off under vacuum at 45° C. The contents were heated to reflux and maintained for 30 minutes at reflux. The reaction mass was cooled to room temperature and stirred for 1 hours at room temperature. The reaction mass was further cooled to 10 to 15° C. and stirred for 1 hour 30 minutes at 0 to 5° C. The solid obtained was collected by filtration and dried at 50 to 55° C. for 7 hours to obtain 145 gm of highly pure olmesartan medoxomil.

Olmesartan medoxomil: 99.6%;
Olmesartan acid impurity: 0.08%;
Trityl olmesartan medoxomil impurity: 0.08%
Methyl olmesartan medoxomil impurity: 0.09%.

1. A process for the preparation of substantially pure 4-(1-hydroxy-1-methylethyl)-2-propyl-1-[[2′-[(N-triphenylmethyl-1H-tetrazole-5-yl)[[1,1′-biphenyl]-4-yl]methyl]-1H-imidazole-5-carboxylic acid (5-methyl-2-oxo-1,3-dioxal-4-yl)methyl ester (trityl olmesartan medoxomil), comprising
adding a solution of trityl olmesartan acid in a solvent selected from a ketonic solvent, dimethylformamide and dimethyl acetate, to a solution of (4-bromoomethyl)-5-methyl-2-oxo-1,3-dioxane (medoxomil) in the, and
isolating substantially pure trityl olmesartan medoxomil.

2. The process according to claim 1, wherein the solvent is a ketonic solvent selected from acetone, methyl ethyl ketone, methyl isobutyl ketone and diethyl ketone.

3. The process according to claim 2, wherein the solvent is acetone.

4. The process according to claim 1, wherein the reaction is carried out at below 65° C.

5. The process according to claim 4, wherein the reaction is carried out at 35 to 65° C.

6. The process according to claim 5, wherein the reaction is carried out at 45 to 55° C.

7. A process for the purification of trityl olmesartan medoxomil, comprising
a. stirring trityl olmesartan medoxomil with a solvent system comprising water and a solvent selected from ether solvent, ester solvent and mixtures thereof to form a suspension; and
b. isolating highly pure trityl olmesartan medoxomil from the suspension.

8. The process according to claim 7, wherein the ether solvent used in step (a) selected from methyl tert-butyl ether, tetrahydrofuran, 1,4-dioxane and diethyl ether.

9. The process according to claim 8, wherein the ether solvent is methyl tert-butyl ether.

10. The process according to claim 7, wherein the ester solvent used in step (a) selected from ethyl acetate, methyl acetate, isopropyl acetate, tert-butyl methyl acetate and ethyl formate.

11. The process according to claim 10, wherein the ester solvent is ethyl acetate.

12. The process according to claim 7, wherein the step (a) is carried out at elevated temperature.

13. The process according to claim 12, wherein the step (a) carried out at above 25° C.

14. The process according to claim 13, wherein the step (a) is carried out at 40 to 90° C.

15. The process according to claim 14, wherein the step (a) is carried out at 45 to 80° C.

16. The process according to claim 7, wherein the water to the solvent is between 4:1 and 1:1.

17. The process according to claim 16, wherein the water to the solvent is between 3:1 and 1.5:1.

18. A process for the purification of olmesartan medoxomil:
a. stirring a suspension or a solution of olmesartan medoxomil with methyl ethyl ketone; and
b. isolating highly pure olmesartan medoxomil from the solution or suspension.

19. The process according to claim 18, wherein the step (a) of the suspension or solution is carried out at elevated temperature.

20. The process according to claim 19, wherein the step (a) is carried out at above 25° C.

21. The process according to claim 20, wherein the step (a) is carried out at 30° C. to reflux temperature of methyl ethyl ketone.
